(19) United States
(12) Patent Application Publication
(10) Pub. No.: US 2020/0281508 A1
(43) Pub. Date: Sep. 10, 2020

(54) HUMAN BODY MOUNTED SENSORS USING MAPPING AND MOTION ANALYSIS

(71) Applicant: Figur8, Inc., Boston, MA (US)

(72) Inventors: Tiegeng Ren, Westford, MA (US); Keith Desrosiers, Foster, RI (US); Nan-Wei Gong, Cambridge, MA (US); Hua Yang, Concord, MA (US)

(73) Assignee: Figur8, Inc., Boston, MA (US)

(21) Appl. No.: 16/823,417

(22) Filed: Mar. 19, 2020

Related U.S. Application Data
(63) Continuation-in-part of application No. 16/745,595, filed on Jan. 17, 2020.
Application No. 16/745,595 is a continuation-in-part of application No. 16/529,851, filed on Aug. 2, 2019.
Application No. 16/529,851 is a continuation-in-part of application No. 15/875,311, filed on Jan. 19, 2018.
Application No. 15/875,311 is a continuation-in-part of application No. 15/271,863, filed on Sep. 21, 2016.
(60) Provisional application No. 62/821,071, filed on Mar. 20, 2019.
(60) Provisional application No. 62/742,222, filed on Oct. 5, 2018.
(60) Provisional application No. 62/714,241, filed on Aug. 3, 2018.
(60) Provisional application No. 62/513,746, filed on Jun. 1, 2017.
(60) Provisional application No. 62/464,443, filed on Feb. 28, 2017.
(60) Provisional application No. 62/448,525, filed on Jan. 20, 2017.
(60) Provisional application No. 62/221,590, filed on Sep. 21, 2015.
(60) Provisional application No. 62/991,113, filed on Mar. 18, 2020.
(60) Provisional application No. 62/821,071, filed on Mar. 20, 2019.

(51) Int. Cl. A61B 5/11; A61B 5/00; G16H 20/30; G01P 15/02; G01C 19/00; G01R 33/02

(52) U.S. Cl. CPC A61B 5/1126; G16H 40/67; A61B 5/1107; A61B 5/0024; A61B 5/4884; A61B 5/4528; A61B 5/4519; A61B 5/7278; A61B 5/4082; G16H 20/30; G01P 15/02; G01C 19/00; G01R 33/02; A61B 2562/0219; A61B 2562/0223; A61B 5/744

(57) ABSTRACT

Disclosed embodiments describe techniques for motion analysis based on human body mounted sensors. The motion analysis is based on human body mounted sensors using mapping and motion analysis. The wearable sensors include inertial measurement sensors, muscle activation sensors, stretch sensors, or linear displacement sensors. Data is obtained from two or more sensors attached to a body part of an individual, where the two or more sensors enable collection of motion data of the body part, and where the two or more sensors include at least one inertial measurement unit (IMU) and at least one sensor determining muscle activation. The data is processed to determine locations of each of the two or more sensors. The locations of each of the two or more sensors are mapped into a coordinate reference system. The mapping is provided to a motion analysis system. Additional data is obtained to further calculate body part motion.

### RELATED APPLICATIONS

This application claims the benefit of U.S. provisional patent applications “Human Body Mounted Sensors with Mapping and Motion Analysis” Ser. No. 62/821,071, filed Mar. 20, 2019 and “Body Part Consistency Pattern Generation Using Motion Analysis” Ser. No. 62/991,113, filed Mar. 18, 2020.

This application is also a continuation-in-part of U.S. patent application “Movement Biomarker Generation Using Body Part Motion Analysis” Ser. No. 16/745,595, filed Jan. 17, 2020, which claims the benefit of U.S. provisional patent application “Human Body Mounted Sensors with Mapping and Motion Analysis” Ser. No. 62/821,071, filed Mar. 20, 2019.

The U.S. patent application “Movement Biomarker Generation Using Body Part Motion Analysis” Ser. No. 16/745,595, filed Jan. 17, 2020 is also a continuation-in-part of U.S. patent application “Body Part Motion Analysis Using Kinematics” Ser. No. 16/529,851, filed Aug. 2, 2019, which claims the benefit of U.S. provisional patent application “Body Part Motion Analysis Using Kinematics” Ser. No. 62/714,241, filed Aug. 3, 2018, “Wearable Sensors with Ergonomic Assessment Metric Usage” Ser. No. 62/742,222, filed Oct. 5, 2018, and “Human Body Mounted Sensors with Mapping and Motion Analysis” Ser. No. 62/821,071, filed Mar. 20, 2019.

The U.S. patent application “Body Part Motion Analysis Using Kinematics” Ser. No. 16/529,851, filed Aug. 2, 2019 is also a continuation-in-part of U.S. patent application “Body Part Deformation Analysis Using Wearable Body Sensors” Ser. No. 15/875,311, filed Jan. 19, 2018, which claims the benefit of U.S. provisional patent applications “Body Part Deformation Analysis with Wearable Body Sensors” Ser. No. 62/448,525, filed Jan. 20, 2017, “Body Part Deformation Analysis using Wearable Body Sensors” Ser. No. 62/464,443, filed Feb. 28, 2017, and “Body Part Motion Analysis with Wearable Sensors” Ser. No. 62/513,746, filed Jun. 1, 2017.

The U.S. patent application “Body Part Deformation Analysis Using Wearable Body Sensors” Ser. No. 15/875,311, filed Jan. 19, 2018 is also a continuation-in-part of U.S. patent application “Electronic Fabric for Shape Measurement” Ser. No. 15/271,863, filed Sep. 21, 2016, which claims the benefit of U.S. provisional patent application “Electronic Fabric for Shape Measurement” Ser. No. 62/221,590, filed Sep. 21, 2015.

Each of the foregoing applications is hereby incorporated by reference in its entirety.

### FIELD OF ART

This application relates generally to motion analysis, and more particularly to human body mounted sensors using mapping and motion analysis.

### BACKGROUND

The detection and measurement of motion and deformation of a given shape are of keen interest in a variety of research, computational, manufacturing, and other technical and professional fields. The accurate measurement of the motion and the deformation of the shape directly applies to machine vision, industrial automation, scientific biomechanics research, medical treatment, and three-dimensional animation, among many others. The types of shapes that are measured include objects of interest, manufactured parts, body parts, etc. The measurements can be used for object differentiation, where the object differentiation is based on material, size, shape, location, or cost, among many other parameters. When the shape being measured is a portion of a body such as the human body, then measurement of the shape has further applications in industries such as healthcare, sports, fashion, or 3D animation for entertainment and gaming. Accurate shape measurement can be used to obtain critical data such as personal medical information and can be used to design proper medical treatments and equipment such as prostheses. Proper medical treatments are essential for comfort, safety, and therapeutic outcomes for an individual.

Clinical settings demand accurate and precise human body measurements, which are notoriously difficult to obtain. For example, consider a relatively simple, static, volumetric body part measurement, such as measuring the volume of fluid buildup in a limb caused by lymphedema. This is typically a manual process where a tape measure is often used by a clinical professional to make body measurements. First the limb is marked along a longitudinal axis using the tape measure and a marking pen. An appropriate gradation, say every 1 cm, is marked. Next, a transverse circumference is measured at every gradation and recorded. The transverse circumferential measurements are repeated along the desired length of the limb. At a subsequent clinical visit, perhaps one week or one month later, the measurements are taken again. Total limb volume V can be approximated by assuming a step-wise linear series of cylindrical disks. The volume V can be expressed as the area A of each transverse cross-section (where A=C²/4π, and where C is the measured circumference) times the height h of each gradation, and then all of the cylindrical disk volumes can be summed into the total volume. In this way, lymphedema progression and/or treatment effectiveness can be monitored. Unfortunately, even though this measurement example is a relatively simple one involving a static measurement of a non-moving body part, the typical clinical approach is fraught with inconsistencies and opportunities for human error. A different person may be making the measurements. Inconsistent pressure may be applied when measuring the circumference. The tip of the marking pen can be several mm wide. Subtle limb shape changes, whether related to lymphedema or not, may greatly affect the accuracy of the estimated volumetric model calculation. Many such difficulties exist for making even this relatively simple, static body part measurement.

While making static body part measurements is very difficult, the measurement of moving body parts, such as a joint, is far more difficult to achieve. Body part joint movement is three-dimensional, and the movement happens in real-time, that is, non-statically. By necessity, the body part joint is moving when a measurement needs to be taken. Body part joint measurements can involve different deformations along multiple axes. Multiple measurements of a repetitive motion may be required. Measurements may need to be made while the body part is under a load condition or under nominal conditions. All of these variables present an additional layer of variation that makes measurement difficult. Added to this complexity is the fact that body part joints are connected to other body part joints, which further complicates measurement and analysis of shape motion and deformation. Accordingly, accurate measurement and analysis of body part motion is critical.

### SUMMARY

Motion analysis is based on human body mounted sensors using mapping and motion analysis. The ability to analyze a body part that is in motion depends directly on the measurement fidelity of the motion of the body part. The successful analysis of the motion of a body part is critically linked to the accurate measurement of the motion. The analysis of the motion can be used for medical diagnostics such as extent of an injury or disease, for measuring medical treatment efficacy, or for sport performance enhancement. Techniques for body part motion analysis based on human body mounted sensors using mapping and motion analysis are disclosed. Two or more sensors, such as inertial measurement units (IMUs), muscle activation sensors, or stretch sensors, are applied to a body part of an individual. The electrical characteristics of a sensor can change as the sensor is moved, displaced, stretched etc. The electrical characteristics of an IMU change as the IMU accelerates, rotates, or changes position. The sensors are attachable to a body part. Tape, wrap, or a garment can be applied to the body part, and the sensors can be attached to the tape, wrap, or garment using hooks. The tape can be a specialized tape such as physical therapy tape, surgical tape, therapeutic kinesiology tape, and so on. One or more strips of tape can be attached to the body part. The one or more strips of tape can be attached in various configurations. The body part can include one or more of a knee, shoulder, elbow, wrist, hand, finger, thumb, ankle, foot, toe, hip, torso, spine, arm, leg, neck, jaw, head, or back. Data, including changes in electrical information, is obtained from the two or more sensors, where the changes in electrical information are caused by motion of the body part. The obtained data is processed to determine locations of each of the two or more sensors. The sensors can include integrated sensors. The sensors can form a network of sensors. The locations of each of the two or more sensors are mapped into a coordinate reference system. The coordinate reference system can include spherical coordinates, cylindrical coordinates, Cartesian coordinates, etc. The mapping is provided to a motion analysis system. The motion analysis system can calculate the motion of the body part based on the mapping and additional data that can be obtained from the two or more sensors. The motion of the body part can be displayed based on the mapping, the additional data, and the providing. The displaying can include displaying an animation of the motion.

A computer-implemented method for motion analysis is disclosed comprising: obtaining data from two or more sensors attached to a body part of an individual, wherein the two or more sensors enable collection of motion data of the body part, and wherein the two or more sensors include at least one inertial measurement unit (IMU) and at least one sensor determining muscle activation; processing the data to determine locations of each of the two or more sensors; mapping the locations of each of the two or more sensors into a coordinate reference system; and providing the mapping to a motion analysis system. Additional data is obtained from the two or more sensors, wherein the additional data reflects motion of the body part of the individual. The motion of the body part is calculated based on the mapping and the additional data. The motion of the body part is displayed based on the mapping, the additional data, and the providing. The displaying comprises displaying an animation of the motion.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for human body mounted sensors with mapping and motion analysis.

FIG. 2 is a flow diagram for augmenting.

FIG. 3 is a block diagram for mapping and motion analysis.

FIG. 4 is a system diagram for body part motion analysis with wearable sensors.

FIG. 5 shows an apparatus for attachment to tape on one or more body parts.

FIG. 6 illustrates sensor configuration.

FIG. 7A shows shoulder motion.

FIG. 7B shows data collection from shoulders.

FIG. 7C shows sensor positions for data collection from shoulder.

FIG. 8 illustrates detail of a capacitive sensor.

FIG. 9 shows an arm motion display.

FIG. 10 illustrates 2-D projection of muscle and motion patterns.

FIG. 11 is a system diagram for human body mounted sensors with mapping and motion analysis.

### DETAILED DESCRIPTION

Techniques for motion analysis based on human body mounted sensors using mapping and motion analysis are disclosed. Data is obtained from two or more sensors attached to a body part of an individual. Sensors, such as wearable inertial measurement unit (IMU) sensors, muscle activation sensors, and linear displacement sensors, can be applied to a body part of an individual. The body part can include the body, the head, a limb, or any other designated portion of a body part. The sensors can be applied to symmetric body parts such as shoulders, elbows, hips, knees, arms, legs, etc. The sensors can be attached to a fabric which can be attached to a body part. The fabric can include tape, a strap, a woven fabric, a knitted fabric, a garment, etc. The tape can be a specialized tape such as a physical therapy tape, surgical tape, therapeutic kinesiology tape, and the like. The attached sensors can be used to measure various parameters relating to movement of the body part. The measurement of body part can be used to perform motion analysis; to evaluate a similar body part; to evaluate symmetric operation of similar body parts; to perform micro-expression movement evaluations; to evaluate angle, force, and torque of a body part; and the like. The body part can include one or more segments of the body such as the torso, the head, or a limb, or can comprise a joint, a muscle group, or some other designated portion of a body part.

Data, including data relating to electrical characteristics of a sensor, can be obtained. The electrical characteristics of a sensor, such as an IMU, a wearable muscle activation sensor, or a linear displacement sensor, change as the sensor moves, stretches, deforms, etc. As a sensor stretches, for example, muscle contraction or muscle activity over a time period can be determined for the body part to which the one or more sensors are attached. The electrical information can include changes in capacitance, resistance, impedance, inductance, etc. Location of each of the two or more sensors coupled to the human body can be determined by processing the obtained data. Location can include a position on the human body, coordinates within a three-dimensional space, and the like. The location of each of the two or more sensors can be mapped into a coordinate reference system. The coordinate reference system can be based on a spherical coordinate system, a cylindrical coordinate system, a two-dimensional representation, and so on. Motion of the human body can be calculated based on the mapping. The motion can include movement within a space, movement of limbs or joints, movement relative to a symmetric limb or joint, etc. A movement signature, a movement parameter, etc., can be determined based on the calculating of the motion. The movement signature can be used to compare movement of a limb to its symmetric limb, to measure progress of therapy or recovery, to identify possible movement problems, and so on. In embodiments, movement signature is used to analyze a movement disorder of the human body. A movement disorder of the human body can include a disease such as Parkinson's disease, results of a stroke, neurologic problems, etc. The motion of the human body can be displayed based on the calculating. The display can include a graph, a video clip, etc. In embodiments, the displaying includes displaying an animation of the motion.

Traditional inertial unit-based measurement systems attempt to infer the “absolute” location of a certain point of interest by integrating an acceleration reading in a 3D space. However, the accuracy of such an approach is significantly limited by both sampling rate issues and the fidelity of the on-board accelerometer. Drift is one of many problems that is frequently encountered by IMU-based solutions. Drift causes an error in location distance between the actual location of an object and the calculated/observed location read by the IMU. The drift error results from the accumulative error based on the calculations and worsens over time. In contrast, the technique used here employs a variety of sensors to perform measurement. This approach is immune from the accumulative error. Body movement such as that represented by muscle contraction output and mechanical displacement measured across a joint, can be accurately represented in a 3D space over time.

Disclosed techniques address motion analysis based on human body mounted sensors using mapping and motion analysis. In embodiments, tape, such as physical therapy tape, therapeutic kinesiology tape, surgical tape, etc., can be applied to a body part. In other embodiments, the body part can be wrapped, placed in a garment, etc. The body part can include one or more of a limb, a joint, or a muscle. In embodiments, the tape can be applied to symmetric body parts such as left quadricep and right quadricep, left bicep and right bicep, etc. One or more sensors can be affixed to the tape that is applied to a body part. The attaching of the one or more sensors to the tape, wrap, garment, etc., can be accomplished using hooks, a hook and loop technique, fasteners, clips, bands, connectors, and so on. The one or more sensors that can be applied can provide electrical information which, when analyzed, can be used to compute human body motion. The human body motion over a time period can be calculated.

Techniques for motion analysis can be used for body analysis. The body analysis can include tracking symmetric body parts as the body parts are moved. The movement of the body parts can be related to tracking body part motion, body part diagnosis, body part test, body part therapy, and so on. The body part motion analysis can include acceleration and orientation information. The acceleration and orientation information relating to a body part can be collected by a six-axis or a nine-axis inertial measurement unit (IMU). The six-axis IMU can include acceleration and rotation, and the nine-axis IMU can include acceleration, rotation, and absolute direction information. A wearable muscle activity sensor or a linear displacement sensor can be used to determine motion based on stretch. The wearable muscle activity sensor or the linear displacement sensor can include an electroactive polymer.

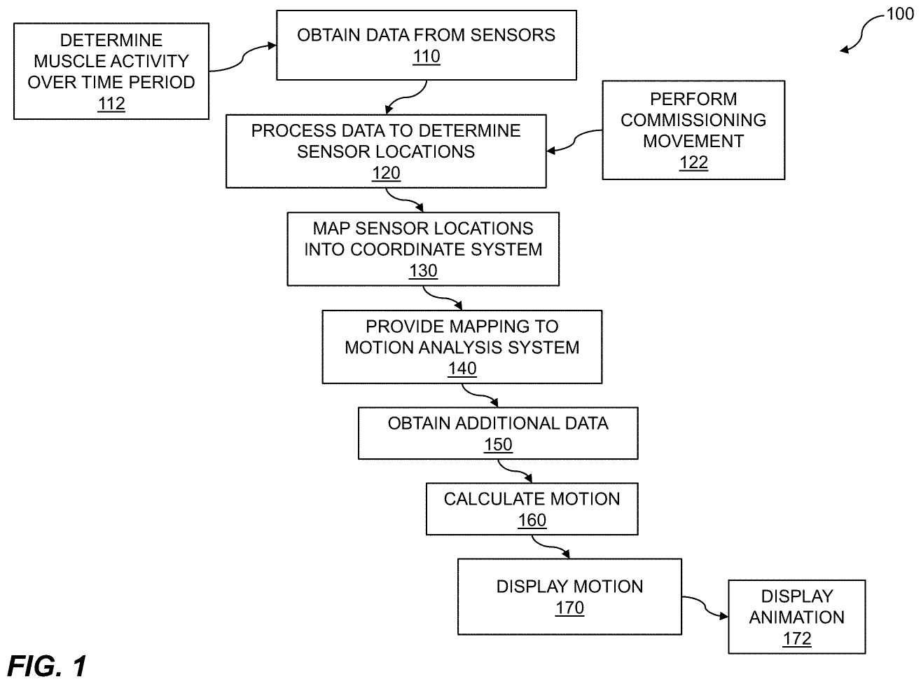

FIG. 1 is a flow diagram for human body mounted sensors using mapping and motion analysis. Two or more human body wearable sensors are used to obtain muscle activity data from a body part of an individual, where the muscle activity data includes electrical information related to the muscle activity or to the physical output of a muscle contraction. A location for each of the two or more sensors coupled to the human body is determined. The location can include a position on the body, a location within a space, and so on. The location of each of the plurality of sensors is mapped into a coordinate reference system, where the coordinate reference system can include spherical coordinates, cylindrical coordinates, Cartesian three-dimensional or two-dimensional representations, and the like. Translation can be performed to convert from one coordinate representation to another coordinate representation. The mapping is provided to a motion analysis system. Motion of the human body is calculated by the motional analysis system based on the mapping. The calculated motion can be used to determine health of a limb or joint, symmetry or motion between symmetric limbs or joints, etc. The calculating motion can be used to assess the stability of a body part, the range of motion for a joint, and the like. The calculating motion can be used to determine ergonomic factors such as body position, posture, or symmetry; to make recommendations to enhance sports performance; to determine medical diagnoses; to propose treatments for injuries; to propose therapies; and so on.

The flow 100 includes obtaining data from two or more sensors coupled to a human body 110, where the two or more sensors enable collection of motion data of the body part. The human body mounted sensors can be attached to tape, where the tape can include physical therapy tape or therapeutic kinesiology tape, or other tape. The human body mounted sensors can be attached to a woven material, a garment, etc. The tape, woven material, garment, and the like, can be applied to, wrapped around, or worn on a body part. Various types of sensors may be coupled to the human body. In embodiments, the two or more sensors include at least one inertial measurement unit (IMU) and at least one sensor determining muscle activation. An IMU can be used to measure specific (e.g. non-gravitational) force, angular rate of rotation, and in embodiments, magnetic field. An IMU can include an accelerometer and a gyroscope, where the accelerometer can measure acceleration, and the gyroscope can determine angular rate. In other embodiments, the IMU can include a magnetometer. The magnetometer can be used to determine direction such as a compass bearing. A sensor that can be coupled to the human body can include two or more sensors. In embodiments, at least one of the two or more sensors can include an integrated stretch sensor and IMU. The integrated sensors can include muscle activation sensors, physiological sensors, optical sensors, etc. The sensors can communicate with each other. In embodiments, the two or more sensors comprise a network of sensors. The network of sensors can be based on a wired network, a wireless network such as a Bluetooth™ network, etc.

Further embodiments include obtaining data from two or more muscle activation sensors coupled to the human body. A muscle activation sensor can be used to measure signals such as electrical signals resulting from muscle activity. Muscle activity can include muscle activation, contraction, relaxation, etc. In embodiments, a muscle activation sensor from the two or more muscle activity sensors is coupled to a sensor from the two or more sensors coupled to the human body. Other sensors can be included within the two or more sensors. The muscle activation sensor can include a stretch sensor or a linear displacement sensor. Further embodiments include obtaining data from a linear displacement sensor. A linear displacement sensor can measure displacement of a muscle based on activating the muscle, flexing or unflexing a joint, and the like. Other embodiments include determining a muscle activity over a time period 112 based on the data from the linear displacement sensor.

The flow 100 includes processing the data to determine locations of each of the two or more sensors 120. Each of the two or more sensors is coupled to the human body as described previously. The location of each sensor can be based on the position of the sensor on a body part. The location of the sensor can include various body parts of a human such as the neck or the small of the back; a joint such as a shoulder, elbow, wrist, hip, knee, or ankle; an extremity such as an arm or a leg; and so on. The location of each sensor can be determined within a coordinate space. In embodiments, the locations of the sensors are determined based on data taken while the individual assumes a commissioning pose. The pose can include standing, reaching, sitting, squatting, and the like. In further embodiments, the locations are determined based on data taken while the individual performs a commissioning movement 122. The commissioning movement can include body movement such as walking, running, dancing, swimming, and so on. The pose can be any pose convenient for locating sensors. In a usage example, the pose can include feet together and arms at the sides or “mountain pose”. The pose can include various poses such as feet apart, arms away from the sides, etc. In embodiments, the commissioning movement can include an arms-out squat or a simultaneous arm raise. The commissioning movement can include a balance pose such as standing on one leg. The locations of each of the two or more sensors can be determined based on further body movements. In other embodiments, the locations can be determined based on constrained movements of the human body. The constrained movements of the human body can include limiting degrees of freedom of movement or isolation a body part. In embodiments, the constrained movements can be based on location of hinge joints and ball-and-socket joints of a human body.

The flow 100 includes mapping the locations of each of the two or more sensors into a coordinate reference system 130. The coordinate reference system can be chosen based on convenience of representing a location, computational simplicity, and so on. In embodiments, the coordinate reference system can include spherical coordinates. Spherical coordinates can be useful for determining simple or complex body motion within a three-dimensional space. In other embodiments, the coordinate reference system can include cylindrical coordinates. Cylindrical coordinates can be convenient for determining body motion that includes rotation. In further embodiments, the coordinate reference system can include a two-dimensional or Cartesian representation. Three-dimensional coordinates can be projected onto a two-dimensional plane or representation. A two-dimensional representation can be particularly useful for reducing computational complexity. Translation can be performed to translate a coordinate representation to another coordinate representation.

The flow 100 includes providing the mapping to a motion analysis system 140. Described below and throughout, a motion analysis system can include one or more computers, processors, and so on. The motional analysis system can include cloud-based computing capabilities, mesh computing, etc. The motion analysis system can determine human body motion, and can analyze the motion to identify asymmetry, weakness, injury, or other motion associated with body part motion. In embodiments, a computer system for motion analysis comprises: a memory which stores instructions; one or more processors coupled to the memory wherein the one or more processors, when executing the instructions which are stored, are configured to: obtain data from two or more sensors attached to a body part of an individual, wherein the two or more sensors enable collection of motion data of the body part, and wherein the two or more sensors include at least one inertial measurement unit (IMU) and at least one sensor determining muscle activation; process the data to determine locations of each of the two or more sensors; map the locations of each of the two or more sensors into a coordinate reference system; and provide the mapping to a motion analysis system.

The flow 100 further includes obtaining additional data 150 from the two or more sensors, wherein the additional data reflects motion of the body part of the individual. The additional data can be collected from the two or more sensors contemporaneously with the collection of the first data discussed previously. The additional contemporaneous data can be compared to the first data to determine accuracy of the data such as a “reality check”. The additional data can be collected at a time subsequent to the collection of the first data. The additional data can be compared to the first data to determine changes in motion of the body part or muscle activity over time. The determining of body part motion or muscle activity over time can be used to gauge effectiveness of a therapeutic therapy or exercise, recovery from surgery or injury, progression of a disease or degenerative condition, and so on.

The flow 100 includes calculating the motion 160 of the body part based on the mapping and the additional data. The motion of the human body part can include raising or lowering a limb, flexing a muscle, and the like. The motion of the human body part can include performing exercises such as therapeutic exercises or strengthening exercises, participating in a sport, etc. In embodiments, the calculating can be based on constrained movements of the human body. The constrained movements of the human body can include flexing a single knee, performing a squat, performing a pushup or plank, etc. In embodiments, the constrained movements can be based on location of hinge joints and ball-and-socket joints of the human body. The calculating motion can be based on various sensors such as the IMU. In other embodiments, the calculating motion is further based on the data from the plurality of muscle activity sensors. The muscle activity sensors, IMUs, linear displacement sensors, etc., can be coupled to a sensor.

The flow 100 includes displaying the motion of the body part 170 based on the mapping, the additional data, and the providing. The displaying the motion of the human body can include rendering a graph, point plot, chart, etc. The displaying can include showing an image, a series of images, a video, and the like. The displaying can be accomplished using a computer, a tablet, a smartphone, or other electronic device to which a display is coupled. The motion that is displayed can be based on a movement signature. The movement signature can be determined for a variety of purposes. In a usage example, the movement signature for the movement of the left knee can be compared to the movement signature for the movement of the right knee. The signature for the movement of the left knee can be compared to the signature for the movement of the right knee to determine imbalances, progress of strengthening or healing, etc. The signature for the movement can be used to analyze a movement disorder of the human body. Movement disorders can result from disease, injury, illness, and so on. In embodiments, the movement disorder can include Parkinson's disease. The movement signature can be used to gauge the frequency and magnitude of tremors that can be associated with Parkinson's disease. In other embodiments, the movement disorder can include results of a stroke. The movement signature can include weakness or limited use of one or more muscles, asymmetries between muscle groups on opposite sides of the human body, etc. In embodiments, the displaying comprises displaying an animation 172 of the motion. The animation of the motion can include an avatar, where the avatar can show the movement of a particular body part or body parts. The movement of the avatar can mimic the movement of the human body. The movement of the avatar can be used to show correct, typical, or recommended movement of the body part. The computed movement of the body part can be compared to these “ideal” body part motions. Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

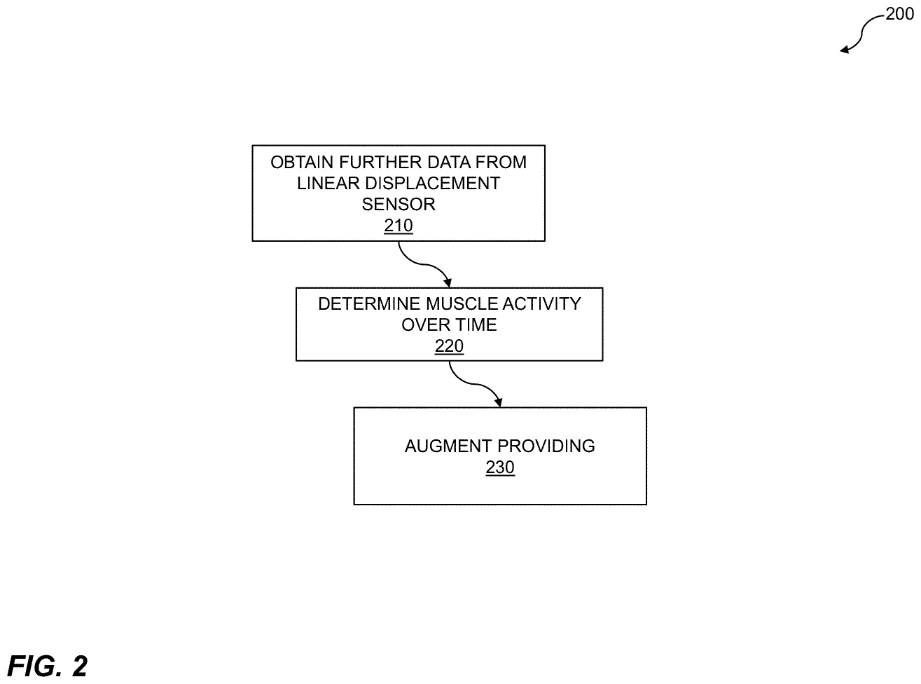

FIG. 2 is a flow diagram for augmenting. Discussed throughout, human body mounted sensors can be used to enable collection of motion data associated with the body part. In embodiments, additional data can be obtained from the sensors. There are many reasons for which additional data can be collected. The additional data can be used to verify collected data, to compare body part motion over a period of time, and so on. The comparison of body part motion data is useful to gauging the effectiveness of a treatment or therapy, to monitor the progression of an illness or disease, and so on. The comparison of body part data can be used to verify that a patient is complying with a treatment or therapy regimen. The collection of additional sensor data enables motion data using mapping and motion analysis.

The flow 200 includes obtaining further data from a linear displacement sensor 210 included in at least one of the two or more sensors. The obtaining further data can include obtaining data from an IMU, a muscle activation sensor, a physiological sensor, an optical sensor, and so on. The obtaining further data can occur at the time the data is collected, at time intervals such as once a week, once a month, quarterly, etc. The flow 200 includes determining a muscle activity over a time period 220 based on the data from the linear displacement sensor. The determining muscle activity over a time period can be used to determine progress of healing from surgery or an injury. The determining muscle activity over a time period can be used to measure the progress of a disease. The determining has other uses as well. The determining muscle activity over a time period can be used to determine whether an individual is complying with treatment or therapy programs. The determining can be used to measure and verify an extent of an injury so that treatment and therapies can be designed based on the extent of the injury rather than the extent reported by an individual prone to exaggeration or underreporting. The flow 200 includes augmenting the providing 230 based on the muscle activity over time. The augmenting the providing can be used to improve the mapping of locations of each of the two or more sensors. The augmenting the providing can be used to provide additional motion data so that the accuracy of the motion analysis computed by the motion analysis system can be improved.

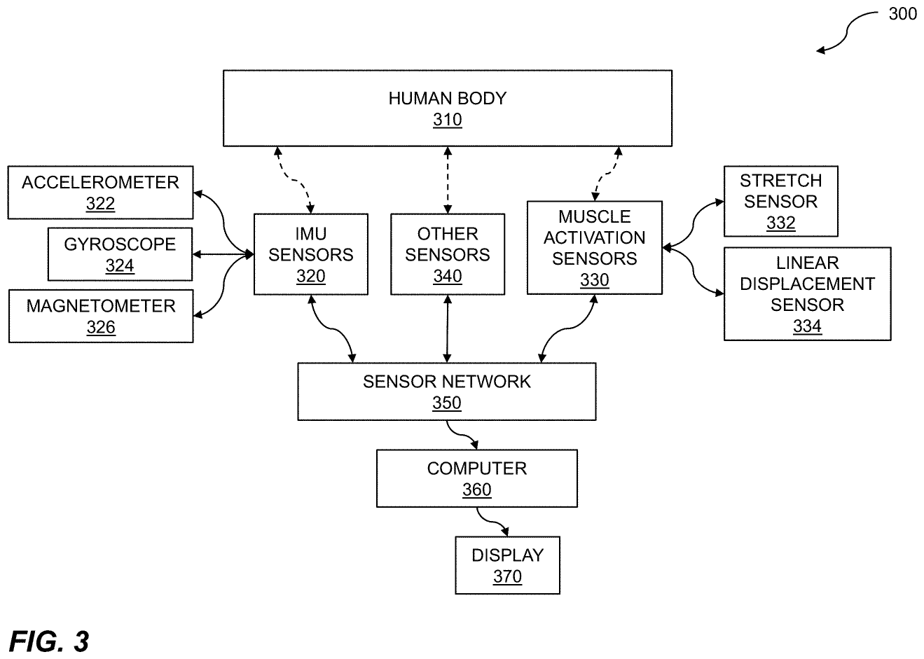

FIG. 3 is a block diagram for mapping and motion analysis 300. Sensors of various types can be used for motion analysis. The sensors can include human body mountable sensors. Data is obtained from two or more sensors coupled to a human body. The sensors can be applied to tape, a wrap, a garment, and so on. The sensors can be included within the tape, wrap, or garment. The data is processed to determine locations of each of the two or more sensors that are attached to the human body. The locations can include symmetric locations such as left and right shoulder, hip, elbow, knee, ankle, etc. The locations can include the cervical spine or neck, lumbar spine or lower back, and the like. The location of each of the two or more sensors is mapped into a coordinate reference system. The coordinate reference system can include spherical coordinates, cylindrical coordinates, Cartesian coordinates, rectangular or a two-dimensional representation, etc. The mapping is provided to a motion analysis system, and the motion of the human body can be calculated based on the mapping.

A human body is represented 310. Various types of sensors can be applied to the human body. The sensors can be applied by coupling the sensors to tape, a garment, a wrap, a strap, or another similar technique. In embodiments, each of the plurality of sensors can include an inertial measurement unit (IMU) 320. An IMU sensor can be used to measure force such as specific (non-gravitational) force, acceleration, or magnetic field. In embodiments, the inertial measurement unit can include an accelerometer 322 and a gyroscope 324. The accelerometer and gyroscope can be used to determine linear velocity, angular rates of rotation, and so on. In other embodiments, the inertial measurement unit can include a magnetometer 326. The magnetometer can be used to measure a magnetic field adjacent to a body. Detection of the magnetic field adjacent to the body can determine a direction of travel, where the direction of travel can include a compass direction. In further embodiments, the plurality of sensors can include muscle activation sensors 330. A muscle activation sensor can be used to sense muscle activation based on muscle motion or micromotion, displacement, deformation, and so on. A muscle activation sensor can be used to detect electrical or other activity within a muscle. The muscle activation sensor can be based on a stretch sensor 332, a linear displacement sensor 334, etc. In other embodiments, the plurality of sensors can include other sensors 340. The other sensors can include physiological sensors, optical sensors, and the like. The physiological or optical sensors can be used to detect heart rate, heart rate variability, respiration rate, etc. In embodiments, the two or more sensors can include a network 350 of sensors. The network of sensors can include a wired network, a wireless network, or a combination of wired and wireless network. Data from a linear displacement sensor or other sensor can be collected over a duration of time. Embodiments include determining a muscle activity over a time period based on the data from the linear displacement sensor.

A computer 360 can be used to process data obtained from the two or more sensors coupled to the human body. The computer can be used to obtain the data, to process the data, to perform operations such as signal processing operations on data, and so on. The operations can include filtering, correlation, compression, interpolation, and so on. The computer can be used to determine a location for each of the two or more sensors. The location can be determined based on a body part to which a given sensor is mounted. The location can be determined within a three-dimensional space, a two-dimensional space, etc. The computer can be used to map the location of each sensor into a coordinate reference system. The coordinate reference system can include spherical coordinates, cylindrical coordinates, Cartesian coordinates for a three-dimensional or a two-dimensional representation, etc. The computer can be used to calculate motion of the human body. The motion can be relative to the human body such as an arm raise, relative to a three-dimensional space such as walking or running, relative to a two-dimensional space such as jumping, etc. The computer can be used for determining a movement signature based on the calculating motion. A movement signature can be useful for comparing movement of an individual at different times, e.g. before and after treatment, therapy, or surgery. The comparing movement can be useful for analysis. Embodiments include using the signature movement to analyze a movement disorder of the human body. A movement disorder can occur due to disease such as Parkinson's disease, illness, a neurological event such as stroke, etc. The computer can be coupled to a display 370. The display can be used to show data, codes, apps, and so on. In embodiments, the display can be used for displaying the motion of the human body based on the calculating. The motion of the human body can be displayed using graphs, a sequence of images, a video clip, and the like. In embodiments, the displaying can include displaying an animation of the motion.

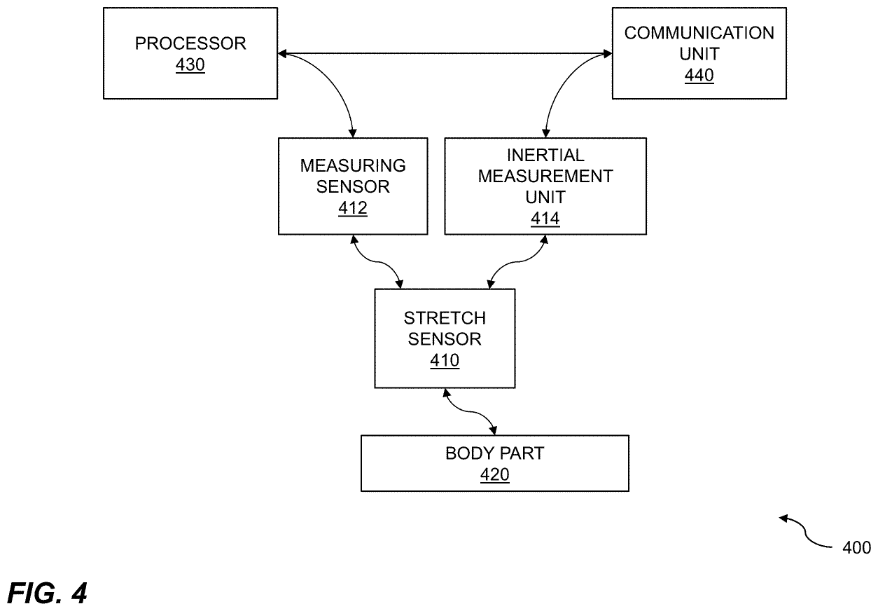

FIG. 4 is a system diagram for body part motion analysis with wearable sensors. Body part motion analysis is based on human body mounted sensors using mapping and motion analysis. The electrical characteristics of a sensor, such as a stretch sensor attached to a body part, change as the sensor stretches. The stretch sensor can include an electroactive polymer or a flexible inductor. The sensor can include an inertial measurement unit (IMU), a muscle activation sensor, a linear displacement sensor, and so on. Data is obtained from two or more sensors coupled to a human body, where the two or more sensors enable collection of motion data of the body part. The data is processed to determine locations of each of the two or more sensors. The locations of each of the two or more sensors are mapped into a coordinate reference system. The mapping is provided to a motion analysis system.

A system diagram for body part motion analysis with wearable sensors is shown. The system diagram 400 includes a stretch sensor 410. Tape can be attached to a body part and a first stretch sensor 410 can be attached to the tape. Connectors, hooks, snaps, Velcro™, and the like can be used to attach the first stretch sensor 410 to the tape. The stretch sensor can include an electroactive polymer. In some embodiments, the stretch sensor includes a capacitive, resistive, or inductive sensor. The tape can include physical therapy tape and therapeutic kinesiology tape, a woven material, etc. The body part 420 to which the stretch sensor is attached can include one or more of a knee, shoulder, elbow, wrist, hand, finger, thumb, ankle, foot, toe, hip, torso, spine, arm, leg, neck, jaw, head, back, and so on. The stretch sensor 410 can be coupled to a wearable measuring sensor 412. The wearable sensor 412 can include two or more body sensors. The wearable sensor can collect electrical information including capacitance, resistance, impedance, inductance, and so on. The stretch sensor 410 can be coupled to an inertial measurement unit (IMU) 414. The inertial measurement unit can capture movement information, attitude information, position information, etc. The measuring sensor 412 can be coupled to a processor 430. The processor 430 can be used for controlling the one or more wearable sensors, for collecting data from the wearable sensors, for analyzing data from the wearable sensors, and so on. The measuring sensor 412 can be coupled to a communication unit 440. The communication unit 440 can provide wired and/or wireless communications between the stretch sensor 410, measuring sensor 412, and/or inertial measurement unit 414, and a receiving unit (not shown). The communication unit 440 can include Ethernet™, Bluetooth™ Wi-Fi, Zigbee™, infrared (IR), and other communications capabilities. The communication unit 440 can send information including movement information, attitude information, position information, and so on.

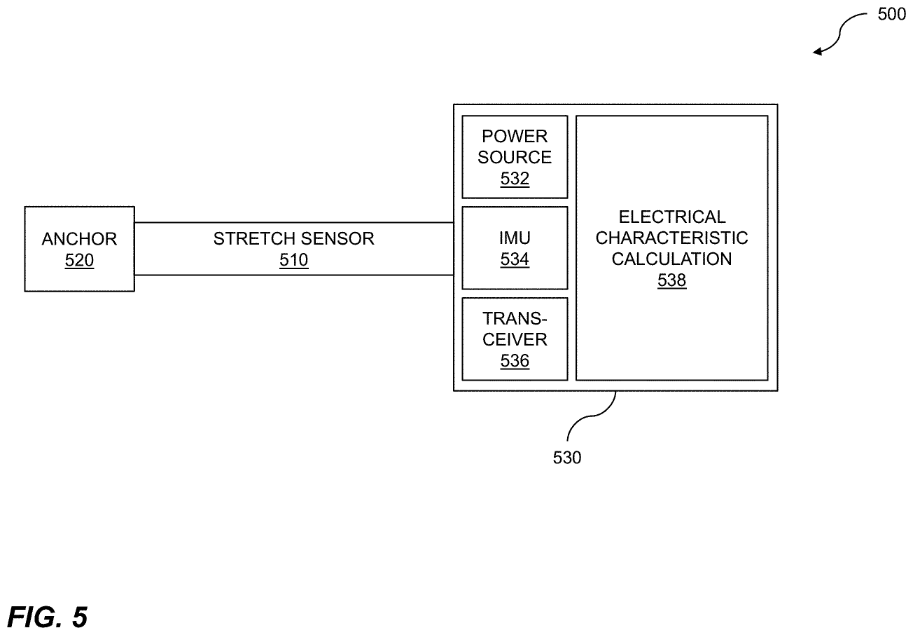

FIG. 5 shows an apparatus for attachment of tape onto one or more body parts of an individual. Body part motion analysis can be based on human body mounted sensors using mapping and motion analysis. The electrical characteristics of a sensor change as the sensor moves, deforms, or stretches in one or more directions. The sensor can include an inertial measurement unit, a muscle activation sensor, a linear displacement sensor, a stretch sensor, and so on. The stretch sensor 500 is attachable to a body part by applying tape to the body part, covering the body part with a garment, and so on. A sensor coupled to the stretch sensor collects changes in electrical characteristics of the sensor based on motion of the body part. Data is obtained from a plurality of sensors coupled to a human body. Location of each of the plurality of sensors coupled to the human body is determined by processing the data. The location of each of the plurality of sensors is mapped into a coordinate reference system, and the mapping is provided to a motion analysis system. Motion of the human body is calculated based on the mapping using a motion analysis system. The calculated motion of the body can be displayed, where the display of the motion can include an animation. An apparatus for attachment to tape on one or more body parts is shown. The apparatus includes a stretch sensor 510. While one stretch sensor is shown, other numbers of stretch sensors can be included. In a usage example, one or more stretch sensors can be attached to symmetrical body parts to compare relative motion of symmetrical joints such as shoulders, hips, or knees. The stretch sensor can include an electroactive polymer. The stretch sensors can be configured in a variety of arrangements such as a t-shape, an offset-t-shape, a w-shape, an x-shape, a spider-shape, and so on. The stretch sensor 510 can be coupled to an anchor 520. The anchor can include hooks or other fasteners, and the anchor can be used to attach the stretch sensor to tape, fabric, and so on. When tape is used, the tape can be attached to the body part and the first stretch sensor can be attached to the tape.

In embodiments, the tape can include physical therapy tape. In other embodiments, the tape can include therapeutic kinesiology tape. The apparatus 500 can include an electrical component 530. The electrical component 530 can be coupled to the stretch sensor 510 and can collect changes in electrical characteristics of the stretch sensor 510. The electrical component 530 can include a power source 532 that can provide power to electrical circuits and can drive the stretch sensor 510. The electrical component can include an electrical characteristic calculation component 538 and an IMU 534. The electrical characteristic calculation component 538 can be used to determine stretch, bulge, displacement, motion, and other physical characteristics based on body part motion. The electrical characteristic calculation component 538 can be used to determine muscle activation and deformation. Muscle activation includes timing and displacement of muscle deformation, and subtle muscle activation analysis can characterize muscle microexpression, which represents detailed muscle movement and timing analysis that can be extremely useful for understanding muscle performance, neuromuscular control, injury, rehabilitation, athletic usage, training, and so on. Muscle microexpression is not detectable by image-based muscle observation, nor is it detectable by IMU-based muscle observation alone, nor is it detectable by a combination of the two.

Movement patterns can include body part movements (e.g., a forearm), body segment movements (e.g., an entire arm), and full body movement patterns (e.g., a golf swing). Muscle contraction movement output magnitudes can be part of a kinematic sequence. The components can be expressed in terms of both magnitude and timing. All of the components can be analyzed, calculated, or inferred by the electrical characteristic calculation component 538. The electrical component 530 can include a transceiver communication unit 536 which can be used to send collected changes in electrical characteristics of the stretch sensor 510 and IMU 534 to a receiving unit (not shown). The transceiver communication unit can include one or more transceivers, where the transceivers can communicate using a variety of wired or wireless techniques. The transceiver communication unit can communicate using Bluetooth™, Zigbee™ or Wi-Fi, near-field communication (NFC) techniques, infrared (IR), etc. In other embodiments, the electrical characteristic calculation component 538 can provide a muscle microexpression summary analysis using the transceiver communication unit 536.

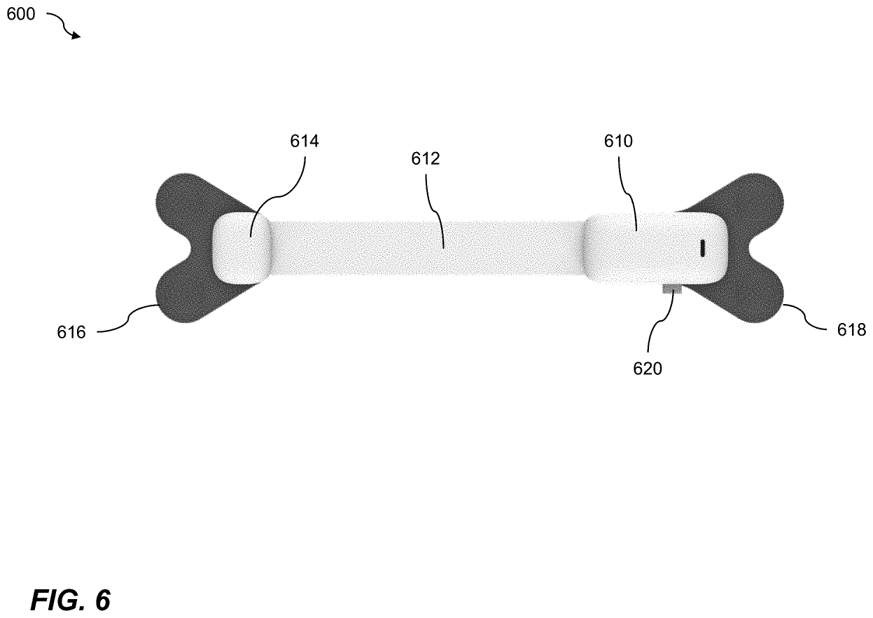

FIG. 6 illustrates sensor configuration. Discussed below and throughout, two or more sensors can be attached to a body part of an individual and can be used to collect motion data of the body part. The data can be processed to determine locations of the sensors, and the locations of the sensors can be mapped into a coordinate system. The mapping of the sensor locations can be provided to a motional analysis system. The sensors that can be attached can include inertial measurement units (IMUs), sensors for detecting muscle activation, stretch sensors, linear displacement sensors, and so on. An IMU can include one or more of an accelerometer, a gyroscope, and a magnetometer. Data is collected from the sensors, where the sensor data includes electrical information based on a micro-expression of movement of the body part. The motion analysis system is used to analyze the electrical information to render a display of the body part motion, an animation of the body part motion, a consistency pattern, or other analysis. The analysis of the body part motion can be used for a clinical evaluation for the individual.

A stretch sensor configuration 600 for attachment to a body part is shown. An IMU, a sensor for determining muscle activation, a stretch sensor, or other sensors, can be used for body part analysis using mapping and motion analysis. The electrical characteristics of a sensor, such as an IMU muscle activation sensor, or stretch sensor, change as the IMU or sensor moves, activates, or stretches, respectively. The electrical characteristics can include resistance, capacitance, inductance, reluctance, and so on. The muscle activations determined by the muscle activation sensor can correspond to movement of a body part to which the sensor is attached. Similarly, motion of an IMU can include acceleration, rotation, or position of the body part. The electrical characteristics of a collector or sensor coupled to the muscle activation motion change based on muscle activation of the body part. A communication unit provides information from the sensor or collector to a receiving unit. The stretch sensor configuration 600 can comprise an apparatus for attachment to tape on a body part. The sensor configuration can include an electrical component 610. The electrical component 610 can be coupled to a stretch sensor 612 and can collect data relating to changes in electrical characteristics of the stretch sensor 612. The electrical component 610 can include a power source that can provide power to electrical circuits and can drive the stretch sensor 612. The power source and circuitry provide other signals such as sinusoids or pulses at various frequencies, AC or DC voltages, etc., that may be required to operate the sensor. The electrical component can include an electrical characteristic calculation component. The electrical characteristic calculation component can be used to determine stretch, bulge, displacement, and other physical characteristics based on body part motion. The electrical component can include a Bluetooth™, Wi-Fi, Zigbee, infrared (IR), or other communication unit which can be used to send collected changes in electrical characteristics of the stretch sensor. While one stretch sensor is shown, other numbers of stretch sensors can be included in a sensor configuration. As stated throughout, additional sensors can be based on IMUs. The electrical component can be coupled to a button 620, switch, or other device for energizing or deenergizing the electrical component.

The stretch sensor 612 can include various materials which can be used to detect or measure stretch. In embodiments, the stretch sensor can include an electroactive polymer. The stretch sensors can be configured in a variety of arrangements such as a t-shape, an offset-t-shape, a w-shape, an x-shape, a spider-shape, and so on. The stretch sensor 612 can be coupled to an anchor 614 for the stretch sensor. The stretch sensor anchor can include hooks, and the anchor can be used to attach the stretch sensor to an anchor 616 and 618. The anchors 616 and 618 can include tape, fabric, wrap, and so on. When tape is used, the tape can be attached to the body part where the first stretch sensor can then be attached to the tape. In embodiments, the tape can include physical therapy tape. In other embodiments, the tape can include therapeutic kinesiology tape.

In other embodiments, the sensor configuration 600 can include a bend sensor. One or more bend sensors can be applied to a body part of an individual and can be used for body part motion analysis. The one or more bend sensors can be used to measure motion of the body part with one or more degrees of freedom. Various techniques can be used to implement a bend sensor such as basing the bend sensor on a compliant capacitive strain sensor. A compliant capacitive strain sensor can comprise a dielectric layer sandwiched between two conducting electrode layers. The dielectric layer and the electrode layers can be based on flexible materials, where the flexible materials can include polymers. The flexible materials such as the polymers can include natural rubber, silicone, acrylic, and so on. Since the polymers can typically be insulators, the electrodes of the bend sensor can be formed by introducing conducting particles into the polymers, where the conducting particles can include nickel, carbon black, and the like. In order for the capacitive strain sensor to be applied to the body part, one or more compliant capacitive strain sensors or other strain sensors can be applied to a material such as tape that can be applied to the body part, a fabric that can enwrap the body part, a garment that can be worn on the body part, and so on. In embodiments, at least one of the two or more sensors comprises a bending sensor.

The compliant capacitive strain sensor can measure strain based on the amount of displacement experienced by the strain sensor. The ability of a compliant capacitive strain sensor to measure strain can be limited by the amount of displacement that can be sustained by the strain sensor before the strain sensor is temporarily or permanently damaged. Excessive strain applied to the strain sensor can cause electrical parameters of the strain sensor, such as the resistance of the strain sensor, to change significantly. The significant change in resistance of the strain sensor can include an “open circuit” (high resistance) resulting from a damaged or destroyed strain sensor.

An application of a sensor, such as the configuration shown, to a body part (e.g. a shoulder) can be used to determine angle measurements for the shoulder. In embodiments, angle measurements can include sagittal plane flexion and extension. In addition to angle measurements for a given body part, muscle function assessment can also be performed. In embodiments, muscle function assessment can include displacement of muscle contraction that can occur during an activity. The activity can include normal physical activity such as yoga and strenuous physical activity such as swimming, rowing, rock climbing, and so on. Peak displacement of a muscle can be based on maximum contraction of key superficial muscle groups. A sensor can be attached to a targeted muscle group, over the location of greatest muscle mass displacement. In addition to peak muscle displacement for muscle function determination, an amount of time required to reach peak muscle contraction can be recorded. Other sensors can be applied to shoulder measurements. In embodiments, the inertial measurement unit (IMU) can be used to track acceleration and orientation of a body part such as a shoulder. Based on measurements collected from the IMU, intersegmental movement can provide information on movement patterns across anatomical joints. The information based on the intersegmental movement provides information on a fluidity of movement and a quality of motion. This information can provide side to side comparison of movement of the anatomical joints for healthy populations in contrast with injured populations.

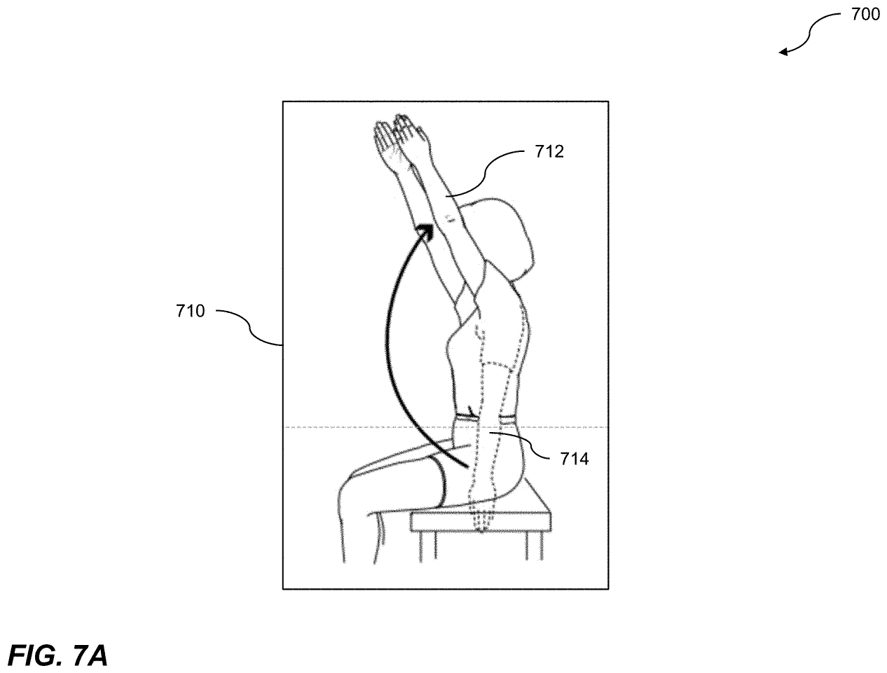

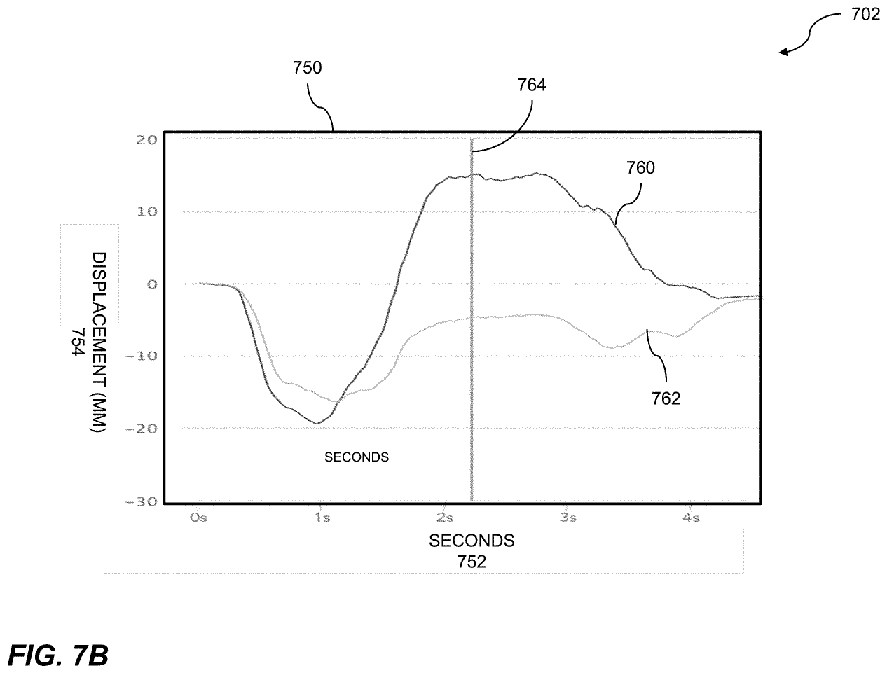

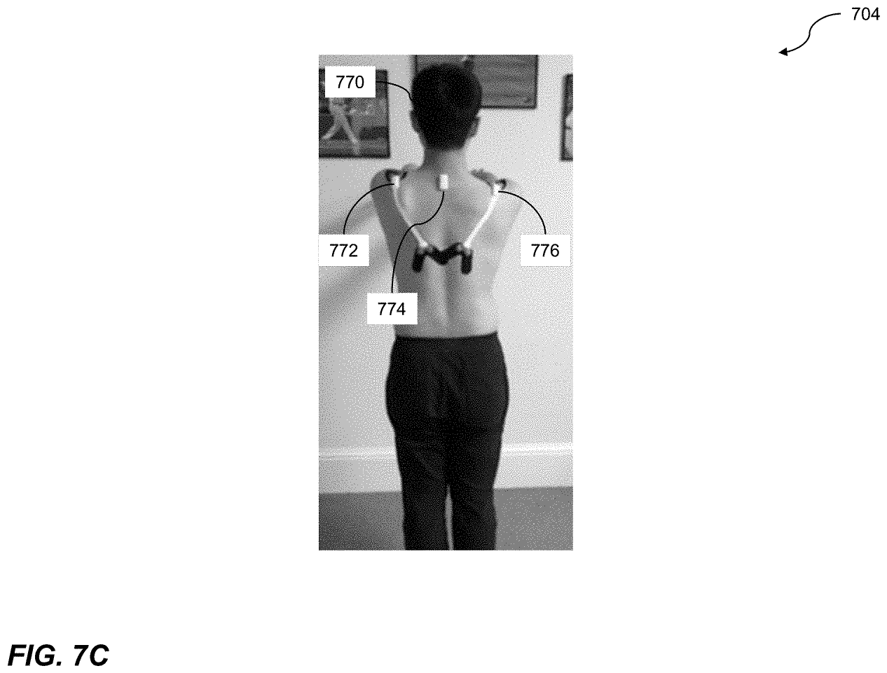

FIG. 7A shows shoulder motion. Two or more sensors such as inertial measurement units (IMUs), muscle activation sensors, stretch sensors, linear displacement sensors, etc. can be used for mapping and motion analysis. The sensors are attached to a body part of an individual. Data from two or more sensors attached to a body part is obtained, where the two or more sensors enable collection of motion data of the body part, and where the two or more sensors include at least one inertial measurement unit (IMU) and at least one sensor determining muscle activation. The data is processed to determine locations of each of the two or more sensors. The locations of each of the two or more sensors are placed into a coordinate reference system. The mapping is provided to a motion analysis system. Additional data can be obtained and further motion of the body part can be calculated. The motion of the body part is based on the mapping, the additional data, and the providing. The displaying can include an animation of the motion.

FIG. 7A shows an example of shoulder motion 700. Two or more sensors such and IMUs and muscle activation sensors can be attached to left and right shoulders of a person 710, where the person can be a surgery patient, an injury patient, a test subject, and so on. The sensors attached to the shoulders of the patient can be used to test for and quantify a severity and a location of a loss of joint stability. In the figure, the patient can raise 712 or lower 714 her left and right arms together as the motion of her left and right shoulders is measured. Embodiments include attaching at least a third sensor to a body part, where in this example, the body part is the spine or centerline at the shoulder region of the individual. The third sensor can be used to enable body part symmetry analysis. The body part symmetry analysis can be used to determine that two body parts, such as left shoulder and right shoulder, are moving properly. In embodiments, the at least third sensor enables an objective measurement of scapular movement.

FIG. 7B shows data collected from shoulders 702. The data can be collected from a single individual, a group of individuals, and so on. The data that can be collected can include data related to a clinical evaluation or diagnosis, an injury, a therapy, and the like. The collected data can include electrical information from an IMU, a sensor determining muscle activation, a stretch sensor, a linear displacement sensor, etc. Plot 750 can show flex return of the left scapula 760 and the flex return of the right scapula 762. The flex return of the left scapula and the flex return of the right scapula can be measured while an individual is executing a movement or a movement performance protocol, performing an action, etc. In embodiments, the movement can be based on assuming a commissioning pose or performing a commissioning movement. The plot 750 can include a time scale in seconds 752 and a displacement in millimeters 754. The plot 750 shows that the percent displacement of the left scapula and the percent displacement of the right scapula differ. The difference in percent displacement can be associated with a surgery, an injury, a disease, and so on. The motion of the body part can be measured as the patient performs an action such as a forward reach activity, an overhead reach activity, a side to side reach activity, a backwards reach activity, and the like. Similar arm reaching activities can be performed with the patient holding one or more weights. A weight can be held in each hand, the weight can be shared between the left hand and the right hand, etc. The vertical line 764 can denote a particular point in time during the reach activity or other activity. For this example, less displacement of the scapula is preferred to more displacement. An increase in displacement of a scapula can indicate impairment, injury, damage, etc.

FIG. 7C shows sensor positions for data collection from shoulder 704. Wearable sensors can be used to collect data relating to a body part of an individual. The data relating to the body part can include position data, motion data, muscle activation data, and the like. The motion data can be analyzed using a motion analysis system to generate a body part consistency pattern, to determine an extent of injury or disease or an amount of recovery, and the like. The body part can include a muscle, a joint, a limb, etc. The data that is collected can include electrical information from the two or more sensors such as an IMU, a muscle activation sensor, a stretch sensor, a linear displacement sensor, or one or more optical sensors. The electrical information can be based on a micro-expression of movement of the body part during a movement performance protocol, a commissioning pose, a commissioning movement such as an arm-out squat or a simultaneous arm raise, etc. A movement performance protocol can include a reach activity. Positionings of sensors for data collection from an individual for shoulder motion are shown. Sensors can be applied to body parts such as shoulders of the individual 770. The sensors can be mounted as shown at the top of the left scapula 772, on a torso centerline 774, at the top of the right scapula 776, and so on. In embodiments, the sensors can be mounted at other positions on the scapulae, at a different point of the torso centerline or spine, on other joints or body parts, and so on. The body parts or locations can include individual body parts such as an arm, shoulder, hip, knee, leg, etc. The sensors can include IMUs, muscle activity or activation sensors, linear displacement or stretch sensors, and so on. The body parts or locations can include symmetrical locations such as left and right shoulder, elbow, hip, or knee; left and right arm or leg; and the like. The sensors which can be mounted can include single-type sensors such as IMU, muscle activity, or linear displacement sensors; or can include combination sensors that can comprise two or more types of sensors. In embodiments, the “combination” sensors can include IMU and muscle activation sensors.

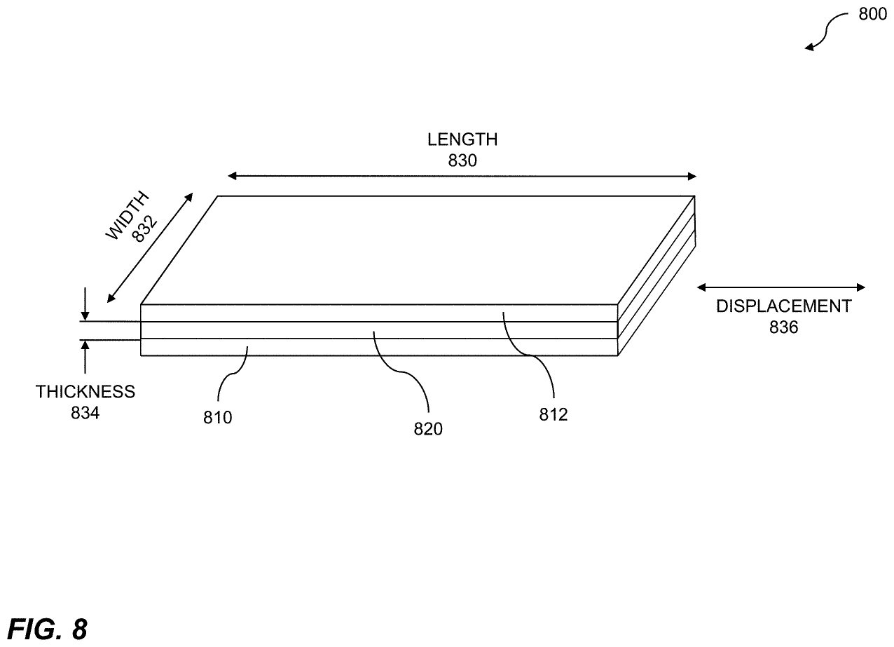

FIG. 8 illustrates detail of a capacitive sensor. Various wearable sensors can include a capacitive sensor. The sensors, including the capacitive sensor, can be used to measure a variety of characteristics, parameters, metrics, etc., that can be associated with the human body. The wearable sensors and the capacitive sensor can include human body mounted sensors using mapping and motion analysis. Data is obtained from two or more sensors coupled to a human body. The data is processed to determine locations of each of the two or more sensors. The locations of each of the two or more sensors are mapped into a coordinate reference system. The mapping is provided to a motion analysis system. Motion of the human body can be calculated based on the mapping. Additional data can be obtained from the two or more sensors. The motion of the body part is displayed based on the mapping, the additional data, and the providing. The display can include an animation of the motion.

Illustration 800 shows a three-dimensional view of a capacitive sensor implementation. The capacitive sensor has a length 830 and a width 832. Embedded between conductive layers 810 and 812 is a dielectric material 820 with thickness 834. The conductive layers 810 and 812 can be attached to a fabric (not shown). The fabric may be a tape such as a therapeutic kinesiology tape, among other such tapes. Therapeutic kinesiology tape often exhibits properties of readily allowing deformation or stretching along only one axis. In this illustration, the length 830 deforms easily, but the width 832 does not readily deform. As the sensor is deformed or stretched along the length 830, a displacement 836 is indicated. However, it is clear that the aforesaid stretching will affect the dielectric material 820 and cause it to become thinner. When one dimension of a three-dimensional solid material with finite volume is expanded, another dimension must contract to maintain the constant, finite volume. The thinning of dielectric material 820 will result in increased capacitance between the conductive layers 810 and 812. The capacitance may be approximated using the general parallel plate capacitor equation C=K*Eo*A/d, where Eo is the permittivity of free space (8.85410⁻¹²), K is the dielectric constant of the material, A is the overlapping surface area of the plates, d is the distance between the plates, and C is capacitance.

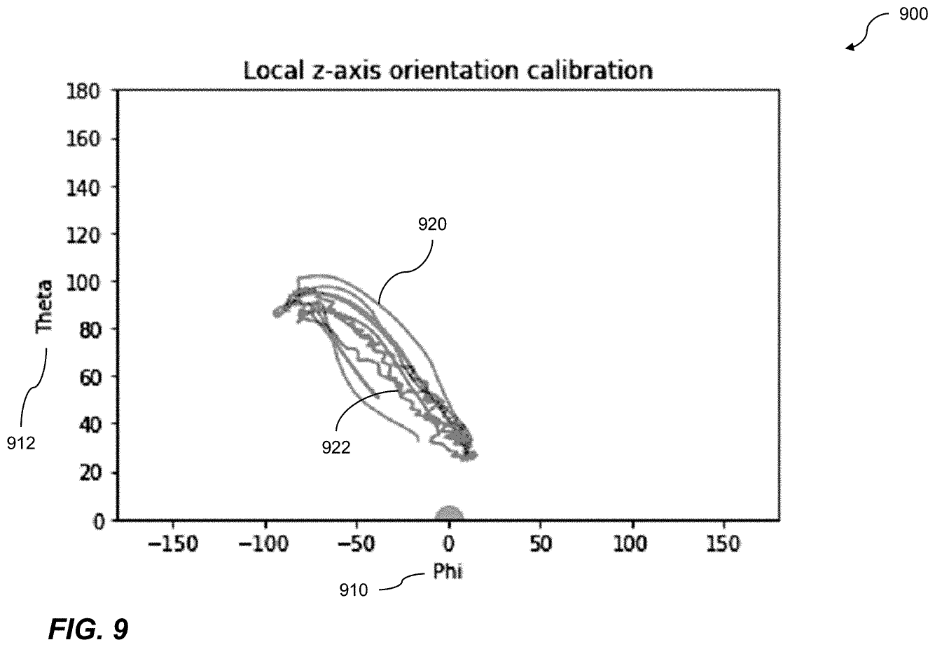

FIG. 9 shows an arm motion display. Sensors that can be attached to a body part of an individual such as an arm can be used to gather data associated with motion of the body part. The locations of the sensors on the body part are mapped, and body part motion analysis is performed based on the mapping. The locations of the sensors can be determined based on data taken while the individual assumes a commissioning pose or performs a commissioning movement. The data can be analyzed using a motion analysis system to determine motion of the body part. Motion of the human body, the body part, etc., can be displayed based on the calculating. The displayed motion can be rendered on an electronic screen such as a display screen coupled to a computer, tablet, smartphone, PDA, and the like. In embodiments, the displaying the motion can include displaying an animation of the motion. The animation of the motion can include an avatar, cartoon, figure, GIF, etc., performing the motion. Motion display supports human body mounted sensors using mapping and motion analysis. Data is obtained from two or more sensors coupled to a human body. The data is processed to determine locations of each of the two or more sensors. The locations of each of the two or more sensors are mapped into a coordinate reference system. The mapping is provided to a motion analysis system, and the motion of the body part is displayed based on the mapping, additional data that can be collected from the sensors, and the providing.

An arm motion display is shown 900. Data obtained from the plurality of sensors can include data from two or more sensors coupled to one arm or both arms of a person. The plot displays angle theta 912 versus angle phi 910. Theta represents 0 to 180 vertical degrees, and phi represents −180 to 180 horizontal degrees. The global reference z is located at phi=0 and theta=0. Plots from arm sensors are shown. Plot 920 can represent data collected from one or more arm sensors. Plot 922 can represent data collected from one or more additional arm sensors. The sensors can include sensors on one arm, sensors on each arm, and so on. The plots 920 and 922 can represent a movement signature based on the calculated motion. Note that plot 922 includes shaking while plot 920 does not. The shaking or “arm shake” can result from strenuous physical activities such as performing pushups or executing a plank pose. The shaking of one arm and the absence of shaking in the other arm can indicate a movement disorder. In embodiments, the movement disorder can include Parkinson's disease. The shaking of one arm and the lack of shaking in the other arm can result from injury, asymmetry between the left arm and the right arm, and so on. In further embodiments, the movement disorder can include results of a stroke.

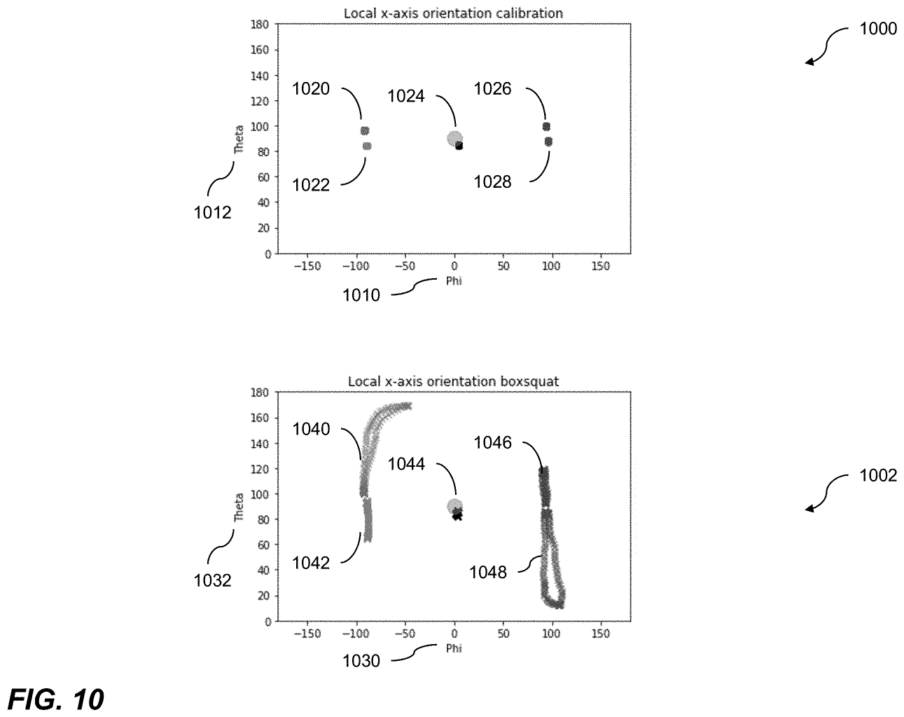

FIG. 10 illustrates 2-D projection of muscle and motion patterns. Two-dimensional projection of muscle and motion patterns can display the motion of a human body. Data rendered in the 2-D projection can be collected from human body mounted sensors with mapping and motion analysis. Data is obtained from two or more sensors attached to a body part of an individual, where the two or more sensors enable collection of motion data of the body part. The two or more sensors can include at least one inertial measurement unit (IMU) and at least one sensor determining muscle activation. The sensors can further include stretch sensors, linear displacement sensors, and so on. The data is processed to determine locations of each of the two or more sensors. The locations of each of the two or more sensors are mapped into a coordinate reference system. The mapping is provided to a motion analysis system. Motion of the human body can be calculated based on the mapping.

To accurately plot one or more muscle and motion patterns, the locations of one or more sensors can be calibrated 1000. This calibration can be accomplished while a human assumes a commissioning pose once sensors are attached to the person. The calibration plot displays angle theta 1012 versus angle phi 1010. Theta represents 0 to 180 vertical degrees, and phi represents −180 to 180 horizontal degrees. The global reference x is located at phi=0 and theta=90. Locations of sensors coupled to a human body are calibrated within the 2-D projection. In the figure, callout 1020 can represent a left quadricep, 1022 can represent a right calf, 1026 can represent a left calf, and 1028 can represent a right quadricep. A sensor which is used for referencing, orientating, aligning, etc. can be used to assist with or supplement determining the location of each sensor. In embodiments, 1024 can represent a sensor placed at the lower back. Reference or other similar sensors can be placed at other locations on the human body. In further embodiments, a sensor can be placed at the back of a neck.

Plots of muscle and motion patterns are shown 1002. The person to whom sensors were applied to left and right quadriceps and left and right calves can perform an exercise, motion, and so on. When the person performs an exercise, the plot can include data collected while the person performs repetitions of the exercise. The plot can include data collected while the person performs the exercise on different dates so that strengthening, recovery from injury, progression of disease, etc., can be determined. In embodiments, the exercise can include a box squat. Data obtained while the person performs a box squat can be collected, mapped, and projected onto two dimensions. As described above, the plot displays angle theta 1032 versus angle phi 1030. Theta represents 0 to 180 vertical degrees, and phi represents −180 to 180 horizontal degrees. The global reference x is located at phi=0 and theta=90. Callout 1040 can represent plotted motion of the left quadricep, 1042 can represent plotted motion of the right calf, 1046 can represent plotted motion of the left calf, and 1048 can represent plotted motion of the right quadricep. Callout 1044 can show displacement of the sensor located at the lower back.

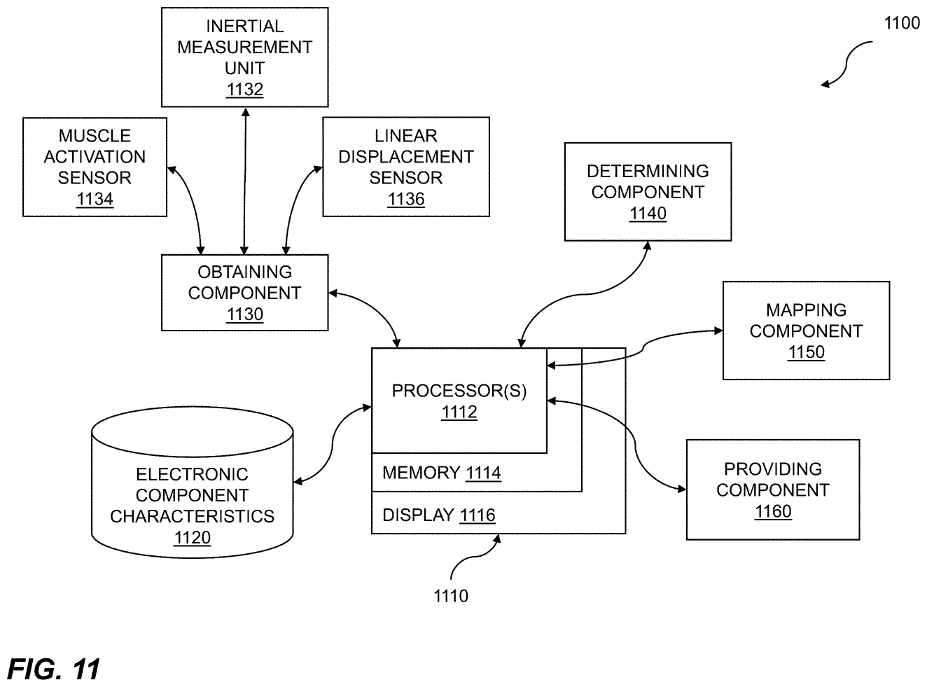

FIG. 11 is a system diagram for human body mounted sensors with mapping and motion analysis. Motion analysis can be calculated based on determining a mapping of one or more sensors and providing the mapping to a motion analysis system. The motion analysis is applied to data obtained from one or more sensors that are attached to a body part. The motion analysis is further based on the mapping. The calculated body motion can be used to determine a body movement signature, where the body movement signature can be used to analyze a movement disorder of the human body. The movement disorder can include Parkinson's disease, results of a stroke, neurological conditions, and the like. The body part motion can include a muscle action such as the action of a bicep, tricep, quadricep, etc.; motion of a body part including a joint(s) such as a lumbothoracic region, shoulder, elbow, hip, knee, ankle, wrist; and so on. Sensors, including body-attachable or wearable sensors, can be used to analyze motion or action of a body part. Various types of sensors can be applied to a body part of an individual, where the application can be accomplished using hooks to attach to tape or straps, suction cups, wraps, garments, safety equipment, and so on. The sensors can include inertial measurement units (IMUs), muscle activation sensors, linear displacement sensors, stretch sensors, and the like. The IMUs can include an accelerometer, a gyroscope, a magnetometer, etc. The electrical characteristics of the linear displacement sensor, the IMU, the muscle activation sensor, etc., can change based on the sensor stretching, accelerating, moving, and the like. The electrical characteristics can include inductance, resistance, or capacitance, where the electrical characteristics change based on movement of the body part. Data is obtained from a plurality of sensors coupled to a human body. Location of each of the plurality of sensors coupled to the human body is determined and mapped. The location of each of the plurality of sensors is mapped into a coordinate reference system. Motion of the human body is calculated based on the mapping.

The system 1100 can include an analysis computer 1110. The analysis computer can include one or more electronic components which can be used to analyze electrical information from wearable sensors. The analysis performed by the analysis computer can calculate motion of the human body. The analysis computer 1110 can comprise one or more processors 1112, a memory 1114 coupled to the one or more processors 1112, and a display 1116. The display 1116 can be configured and disposed to present collected data; sensor location mappings; analysis; intermediate analysis steps; instructions, algorithms, or heuristics; a movement signature; an animation; and so on. In embodiments, one or more processors are coupled to the memory, where the one or more processors, when executing the instructions which are stored, are configured to: obtain data from two or more sensors attached to a body part of an individual, wherein the two or more sensors enable collection of motion data of the body part, and wherein the two or more sensors include at least one inertial measurement unit (IMU) and at least one sensor determining muscle activation; process the data to determine locations of each of the two or more sensors; map the locations of each of the two or more sensors into a coordinate reference system; and provide the mapping to a motion analysis system.

The system 1100 can include electronic component characteristics 1120. The electronic component characteristics can include a library of lookup tables, inductance characteristics, resistance characteristics, capacitance characteristics, functions, algorithms, routines, code segments, apps, and so on, that can be used for the analysis of the electrical information collected from sensors such as IMU sensors, muscle activation sensors, linear displacement sensors, stretch sensors, etc. In a usage example, the electrical component characteristics can include a lookup table that enables mapping of an electrical signal from a linear displacement sensor to millimeters of motion of the body part. The system 1100 can include an obtaining component 1130. The obtaining component can act as an interface between one or more sensors and the analysis computer 1110. The obtaining component can obtain data from one or more human body mountable sensors, where the data can include electrical signals. The electrical signals can be generated by an inertial measurement unit 1132, a wearable muscle activation sensor 1134, a linear displacement sensor 1136, a stretch sensor (not shown), and so on. In further embodiments, the two or more sensors include an integrated stretch sensor and an IMU. The sensors can be in communication with one another. In embodiments, the two or more sensors can include a network of sensors.

The system 1100 can include a determining component 1140. The determining component 1140 can process the data to determine the location of each of the plurality of sensors coupled to the human body. The location of each sensor can include a body part, a muscle or muscle group, and so on. The body part can include an arm, leg, shoulder, hip, torso, etc. In embodiments, the determining further includes determining a muscle activity over a time period based on the data from a linear displacement sensor or other sensor. The determining component can include electronic components or other hardware for determining inductance, resistance, or capacitance. The determining can include determining current, voltage, resistance, capacitance, impedance, and/or inductance. A generating component (not shown) can include hardware for generating direct current and/or alternating current signals used for obtaining resistance and/or capacitance measurements. Typically, the current values are low (e.g. microamperes) and in embodiments, the frequency range includes signals from about 100 hertz to about 1 megahertz.

The system 1100 can include a mapping component 1150. The mapping component can map the locations of each of the two or more sensors into a coordinate reference system. Various coordinate reference systems can be used singly or in combination. The coordinate reference systems can include spherical coordinates, cylindrical coordinates, Cartesian coordinates, and so on. The coordinate reference system can include a two-dimensional representation of the location of each of the plurality of sensors. The system 1100 can include a providing component 1160. The providing component can provide the mapping to a motion analysis system. The motion analysis system can calculate motion of the human body based on the mapping. Further embodiments include obtaining additional data from the two or more sensors, where the additional data reflects motion of the body part of the individual. The obtaining additional data can be accomplished to verify data during a data acquisition session; to obtain data at a later data to determine injury, surgery, or disease recovery; to monitor disease progression, and the like. The motion can include a variety of motions such as voluntary motions or involuntary motions. In embodiments, the calculating can be used for determining a movement signature. The movement signature can be used to identify symmetry between pairs of muscle groups such as left and right biceps. The movement signature can be used to determine asymmetries between the muscle groups, to gauge muscle or joint recovery from injury or surgery, etc. In embodiments, the movement signature can be used to analyze a movement disorder of the human body. A movement disorder of the human body can include Parkinson's disease, results of a stroke, and the like. The system 1100 can include a displaying component (not shown). The displaying component can display the motion of the human body based on the mapping, the additional data, and the providing. In embodiments, the displaying can include displaying an animation of the motion.

The system 1100 can include a computer program product embodied in a non-transitory computer readable medium for motion analysis, the computer program product comprising code which causes one or more processors to perform operations of: obtaining data from two or more sensors attached to a body part of an individual, wherein the two or more sensors enable collection of motion data of the body part, and wherein the two or more sensors include at least one inertial measurement unit (IMU) and at least one sensor determining muscle activation; processing the data to determine locations of each of the two or more sensors; mapping the locations of each of the two or more sensors into a coordinate reference system; and providing the mapping to a motion analysis system.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A computer-implemented method for motion analysis comprising:
obtaining data from two or more sensors attached to a body part of an individual, wherein the two or more sensors enable collection of motion data of the body part, and wherein the two or more sensors include at least one inertial measurement unit (IMU) and at least one sensor determining muscle activation;
processing the data to determine locations of each of the two or more sensors;
mapping the locations of each of the two or more sensors into a coordinate reference system; and
providing the mapping to a motion analysis system.

2. The method of claim 1 further comprising obtaining additional data from the two or more sensors, wherein the additional data reflects motion of the body part of the individual.

3. The method of claim 2 further comprising calculating the motion of the body part based on the mapping and the additional data.

4. The method of claim 3 further comprising displaying the motion of the body part based on the mapping, the additional data, and the providing.

5. The method of claim 4 wherein the displaying comprises displaying an animation of the motion.

6. The method of claim 1 wherein each of the two or more sensors includes an inertial measurement unit (IMU).

7. The method of claim 6 wherein the inertial measurement unit includes an accelerometer and a gyroscope.

8. The method of claim 6 wherein the inertial measurement unit includes a magnetometer.

9. The method of claim 1 wherein at least one of the two or more sensors includes a muscle activation sensor.

10. The method of claim 9 wherein the muscle activation sensor is a stretch sensor or a linear displacement sensor.

11. The method of claim 1 wherein at least one of the two or more sensors comprises an integrated stretch sensor and IMU.

12. The method of claim 1 wherein the two or more sensors comprise a network of sensors.

13. The method of claim 1 wherein the locations are determined based on data taken while the individual assumes a commissioning pose.

14. The method of claim 1 wherein the locations are determined based on data taken while the individual performs a commissioning movement.

15. The method of claim 14 wherein the commissioning movement includes an arms-out squat or a simultaneous arm raise.

16. The method of claim 1 wherein the locations are determined based on constrained movements of a human body.

17. The method of claim 16 wherein the constrained movements are based on location of hinge joints and ball-and-socket joints of the human body.

18. The method of claim 1 wherein the coordinate reference system includes spherical coordinates.

19. The method of claim 1 wherein the coordinate reference system includes cylindrical coordinates.

20. The method of claim 1 wherein the coordinate reference system includes a two-dimensional representation.

21. The method of claim 1 further comprising obtaining further data from a linear displacement sensor included in at least one of the two or more sensors.

22. The method of claim 21 further comprising determining a muscle activity over a time period based on the data from the linear displacement sensor.

23. The method of claim 22 further comprising augmenting the providing based on the muscle activity over time.

24. A computer program product embodied in a non-transitory computer readable medium for motion analysis, the computer program product comprising code which causes one or more processors to perform operations of:
obtaining data from two or more sensors attached to a body part of an individual, wherein the two or more sensors enable collection of motion data of the body part, and wherein the two or more sensors include at least one inertial measurement unit (IMU) and at least one sensor determining muscle activation;
processing the data to determine locations of each of the two or more sensors;
mapping the locations of each of the two or more sensors into a coordinate reference system; and
providing the mapping to a motion analysis system.

25. A computer system for motion analysis comprising:
a memory which stores instructions;
one or more processors coupled to the memory wherein the one or more processors, when executing the instructions which are stored, are configured to:
obtain data from two or more sensors attached to a body part of an individual, wherein the two or more sensors enable collection of motion data of the body part, and wherein the two or more sensors include at least one inertial measurement unit (IMU) and at least one sensor determining muscle activation;
process the data to determine locations of each of the two or more sensors;
map the locations of each of the two or more sensors into a coordinate reference system; and
provide the mapping to a motion analysis system.
